(19) United States
(12) Patent Application Publication
(10) Pub. No.: US 2026/0132433 A1
(43) Pub. Date: May 14, 2026

(54) Expression and Secretion of UV Absorbent Material from Transformed Bacteria

(71) Applicant: UNIVERSITY OF CINCINNATI, Cincinnati, OH (US)

(72) Inventors: Nalinikanth KOTAGIRI, West Chester, OH (US); Tushar Madaan, Cincinnati, OH (US)

(73) Assignee: UNIVERSITY OF CINCINNATI, Cincinnati, OH (US)

(21) Appl. No.: 19/445,118

(22) Filed: Jan. 9, 2026

Related U.S. Application Data
(62) Division of application No. 17/674,161, filed on Feb. 17, 2022, now Pat. No. 12,545,936.
(60) Provisional application No. 63/150,137, filed on Feb. 17, 2021.

(51) Int. Cl. C12P 13/04; A61K 8/99; A61Q 17/00

(52) U.S. Cl. CPC C12P 13/04; A61K 8/99; A61Q 17/00

(57) ABSTRACT

A genetically modified strain of commensal bacteria Staphylococcus epidermidis is provided which produces a material with UV absorbing properties. The material with UV absorbing properties may be a mycosporine-like amino acid such as shinorine.

### CROSS REFERENCE TO RELATED APPLICATIONS

This application claims priority to U.S. Provisional Application Ser. No. 63/150,137, filed Feb. 17, 2021, which application is hereby incorporated by reference in its entirety.

### TECHNICAL FIELD

The present invention relates to a genetically modified bacteria that produces a material with UV absorbing properties.

### BACKGROUND OF THE INVENTION

Environmental stressors on humans and the pressures caused by them are increasing in an ever-changing climate. Stressors such as ultraviolet radiation, heat, osmotic pressure, and desiccation can have several effects on the human body including causing skin cancers, oxidative damage and photoaging. Skin cancers are particularly of acute concern. Among skin cancers, melanoma is a cancer of melanocytes and is expected to affect almost 100,000 people in the year 2019. An aggressive malignancy that tends to metastasize, melanoma is responsible for a majority of skin cancer related deaths in spite of representing 5% of cutaneous malignancies. Therefore, more efficient and effective strategies to minimize UV related damage to skin is required that affords passive protection.

The sun protection and sunless tanners market is a $1.4 billion industry with 3 out of 4 adults using sunscreen, tanning products or sunless tanners. However, the effectiveness of synthetic sunscreens depends on applying generous amounts, inadequate coverage, more frequent applications as much as reapplying every one to two hours, ensuring proper storage since their potency can be destroyed if stored at higher than normal temperature conditions, and other factors that are difficult to control. In addition, consumers are getting increasingly conscious about the safety of sunscreen ingredients as well as the impact of these ingredients on the environment. Recent research has revealed that some synthetic sunscreen components can accumulate in aquatic environments and potentially cause harm by acting as hormone disruptors.

As per the FDA, only two sunscreens (zinc oxide and titanium dioxide) have sufficient safety data. In fact, the FDA in February 2019 said that there is insufficient data on 12 of the 16 approved sunscreen molecules to include them in the “generally recognized as safe and effective” (GRASE) category. Additionally, the FDA has also recommended against the use of aminobenzoic acid (PABA) and trolamine salicylate and have placed them in the non-GRASE category. Only zinc oxide and titanium dioxide were able to get a GRASE designation with FDA suggesting insufficient data for other molecules to make a GRASE determination. The FDA also cited the high systemic availability (including significant concentrations in urine, blood plasma, amniotic fluid, and breast milk) along with insufficient absorption and carcinogenicity data of oxybenzone as a concern against a positive GRASE designation. The FDA is also concerned about potential hormonal disruption with the present set of sunscreen molecules, particularly associated with long-term use.

Certain sunscreen molecules such as oxybenzone, octinoxate, etc. have been found to be toxic to coral reefs, sea urchins, and other marine organisms. As per the National Ocean Service, even GRASE sunscreens such as nano-titanium and zinc oxides can harm marine life. Popular beach destinations such as Palau and Hawaii have already imposed bans on several reef-toxic sunscreens. Therefore, a need still exists for a new method of protecting the skin from UV that is environmentally friendly.

### SUMMARY OF THE INVENTION

The present invention addresses this need by providing a probiotic technology involving engineered commensal bacteria which, when applied to the skin surface, act as living biofactories of sunscreen molecules. These engineered bacteria produce mycosporine-like amino acids (MAAs) which are natural photoprotective molecules produced by marine cyanobacteria. The MAAs produced by commensal skin bacteria multiply on the skin surface and provide sun protection in a sustained manner. Therefore, the problem is solved in an eco-friendly manner since MAAs are sourced from marine life itself.

In one embodiment, the present invention involves a genetically modified strain of commensal bacteria Staphylococcus epidermidis which produces a material with UV absorbing properties. In another embodiment, the material with UV absorbing properties is a mycosporine-like amino acid. In one embodiment, the mycosporine-like amino acid is shinorine. In another embodiment, the bacteria includes the nucleotide sequences shown in SEQ ID NO:6, SEQ ID NO:7, SEQ ID NO:8, and SEQ ID NO:9. In one embodiment, bacteria includes the nucleotide sequence shown in SEQ ID NO:5. In another embodiment, the invention is a topical composition where the bacteria is present in the composition at a concentration of at least 0.1% by weight of the total composition. In one embodiment, the present invention involves a composition including the genetically modified strain of commensal bacteria and a sunscreen.

Another embodiment of the present invention involves a genetically modified strain of bacteria Escherichia coli Nissle 1917 which produces a material with UV absorbing properties. In one embodiment, the material with UV absorbing properties is a mycosporine-like amino acid. In another embodiment, the mycosporine-like amino acid is shinorine. In one embodiment, the bacteria includes the nucleotide sequences shown in SEQ ID NO:1, SEQ ID NO:2, SEQ ID NO:3, and SEQ ID NO:4.

One embodiment of the present invention involves a genetically modified strain of commensal bacteria which produces a material with UV absorbing properties, where the bacteria includes a lysis circuit. In another embodiment, the bacteria undergoes lysis in the presence of increased bacterial density and the absence of UV light.

### BRIEF DESCRIPTION OF THE DRAWINGS

The foregoing summary, as well as the following detailed description of preferred embodiments of the application, will be better understood when read in conjunction with the appended drawings.

FIG. 1 is a schematic of human skin microbiome transformation into “living factories” on skin secreting natural sunscreens for the protection against environmental stressors.

FIG. 2 is a schematic showing release of MAAs from bacteria after UV-induced lysis.

FIG. 3 is a HPLC chromatogram at 333 nm showing Shinorine peak (red arrow) eluting at 15 min timepoint.

FIGS. 4A-D are series of illustrations showing blue light-mediated transcriptional activation and repression of gene expression in bacteria.

FIG. 5 is a nanoLC-MS/MS chromatogram indicating positive identification of MAA producing enzymes by the engineered bacteria.

FIG. 6 is a pair of nanoLC-MS/MS chromatograms indicating positive identification of MAA producing enzymes by the engineered bacteria.

FIG. 7 is a graph showing the significant efficacy of shinorine vs. 10% ZnO 72 h post UV irradiation.

FIG. 8 is a series of images showing cleaved caspase-3 IHC staining of human skin tissues incubated with overnight culture of WT EcN, 10% ZnO, or EcN expressing shinorine 24 h post UV irradiation.

FIG. 9 is a plasmid map of pCN48-Ava3858-3855.

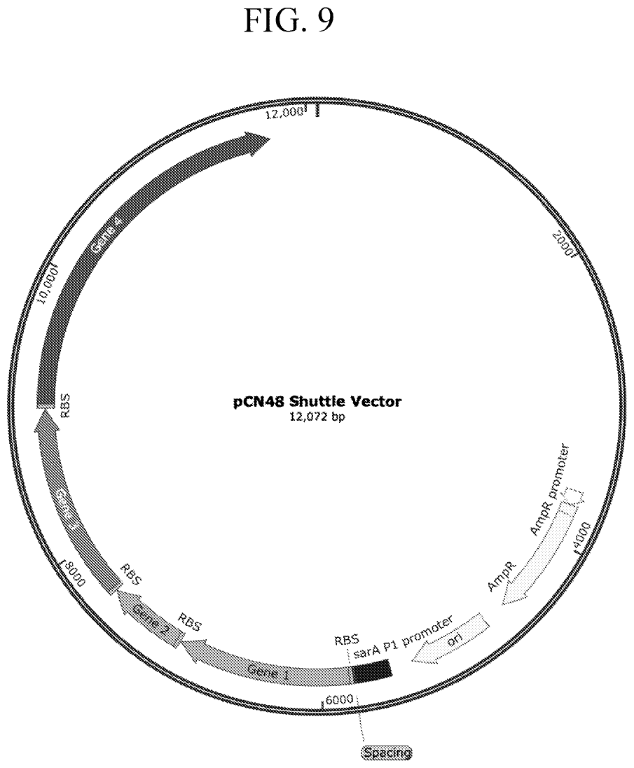

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The term “commensal bacteria” as used herein means a bacteria that lives on or in another organism without causing harm.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Application of sunscreens is crucial to mitigate the risk of skin cancers caused as a result of UV radiation-mediated DNA damage. Studies have shown that a majority of the population does not apply sunscreens correctly and in enough quantity to offer the necessary protection. Therefore, most marketed sunscreens end up offering only about 40% of the promised sun protection factor (SPF) due to improper application. These factors leave users more exposed to solar radiation, significantly limiting the sun shielding offered by these products. Additionally, conventional sunscreens presently available in the market are in the form of lotions, creams, or aerosol sprays that need to be frequently reapplied (usually every two to three hours) which further reduces their compliance and increases risks. The present invention introduces a probiotic sunscreen platform which will form an invisible layer of sun protection on the skin surface.

The human skin is the largest organ in the human body and acts as the interface between the insides of the body and the external environment. The exposed part of the skin is composed of entirely dead elements, including epidermis and hair. Incidentally, the only living element on the surface of the skin is the microbiome. The microbiome has an inherent ability to replenish itself, with some bacteria having doubling times as short as 20 minutes. Recent advancements in genetic engineering and synthetic biology have transformed the field and it's now possible to introduce a diverse array of genetically encoded proteins, drugs, enzymes etc into bacteria as plug-and-play systems. The skin microbiome is a vast and underexplored component of the healthy human body and its importance is gradually being appreciated by researchers and dermatologists worldwide.

By genetically engineering bacteria found in healthy human skin, the present invention presents an unprecedented probiotic sunscreen technology that has not been available before. In addition to being novel, the developed technology is the first of its kind to offer sunscreen protection commensurate with the exposure to dermal exposure to sunlight. The developed platform has the potential to be used for numerous parallel dermatological applications.

### MAAs

Mycosporine-like amino acids (MAAs) are natural ultraviolet radiation absorbing metabolites produced by marine microorganisms such as cyanobacteria and other algae. One of the most popular MAAs is Shinorine, which has been used as a constituent of sunscreen formulations in the European Union such as “Helionori” and “Helioguard 365.” MAAs are photoprotective and are commonly referred to as “microbial sunscreen.” They have potent Ultraviolet-A (UV-A) and UV-B absorbing properties along with anti-oxidant characteristics. Additionally, certain MAAs such as mycosporine-glycine and mycosporine-taurine possess significant singlet oxygen scavenging property. They also perform other protective functions in their parent organisms such as protecting against oxidative stress, desiccation, and osmotic stress. UV irradiation promotes the production of MAAs in cyanobacteria. MAAs such as shinorine have also been found to protect against abiotic stress factors such as salinity, dessication, and heat. However, the yield of shinorine, which comes from red algae gathered from the sea, can vary seasonally and geographically, limiting supply.

S. epidermidis

The present invention involves the creation of genetically engineered bacteria, commonly found on the human skin, for production of these molecules. The human skin is home to several species of commensal or non-pathogenic bacteria and among them is Staphylococcus epidermidis. Interestingly, S. epidermidis possesses genes and precursors involved in the biosynthesis of shinorine, and therefore can be a suitable host for production of shinorine through genetic engineering. These genetically engineered strains of S. epidermidis can be applied on the skin surface to provide both short term as well as long term protection as these bacteria would as living factories on the skin surface constantly generating shinorine on-demand.

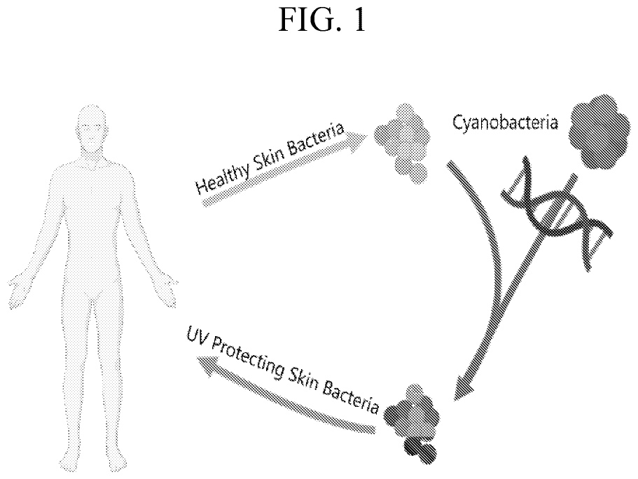

The present invention hijacks the gene cluster from cyanobacteria and engineers S. epidermidis to synthesize MAAs in a regulated manner (FIG. 1). The developed genetically engineered strains of S. epidermidis can be applied on the skin surface to provide both short term as well as long term protection, as these bacteria serve as “living factories” on the skin surface constantly generating Shinorine on-demand.

While S. epidermidis was evaluated as the bacterial template for the “living factory” design, other skin commensal microbiota that might also be effective were screened and identified. For example, commensal bacteria belonging to families, such as Acinetobacter (Moraxellaceae) spp., Bacteroidetes and Proteobacteria spp. are also found abundantly and on specific areas of the skin, thus affording the ability to customize this technology to adapt to heterogeneous body and skin constitutions.

There are several optogenetic response elements (ranging from UV-B to far red) to activate the promoters associated with Shinorine. Bacterial promoters such as recA, lexA, etc. which are part of the robust bacterial SOS response upregulate gene transcription upon detection of DNA damage (e.g., by UV light). Understanding these response elements allows us to respond to UV-B or visible light, given that sunlight is composed of both, to activate and regulate production of Shinorine in a time and intensity sensitive fashion.

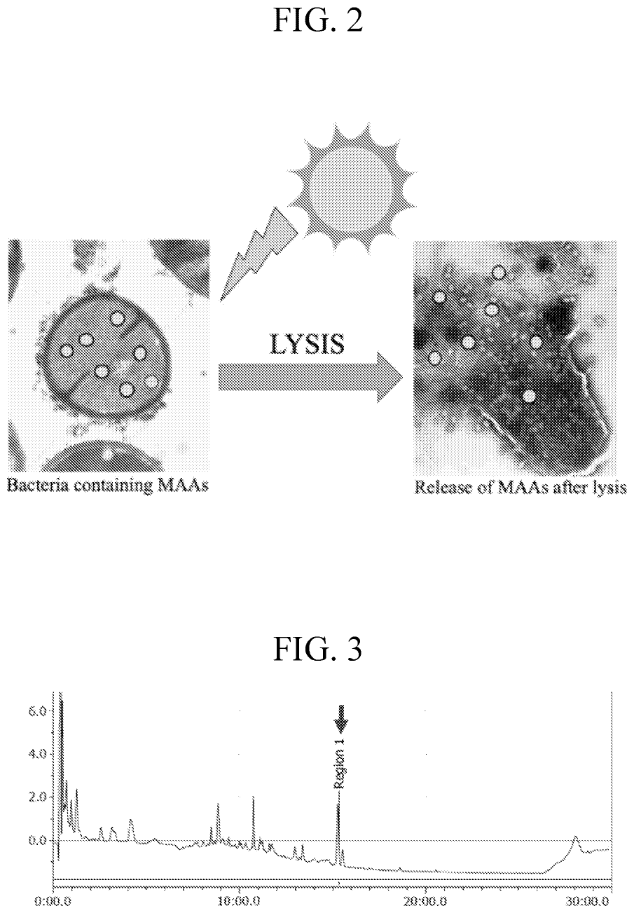

To regulate the bacterial population and potentially release Shinorine extracellularly in significant quantities, in some embodiments, the bacteria is programmed with a lysis circuit to undergo quorum sensing based lysis when high concentrations are reached due to increased bacterial density as well as in the absence of UV light (FIG. 2). With this system, bacterial lysis can be observed in the dark, yet viability is preserved in the presence of UV light and release of sunscreen compounds is commensurate with UV exposure.

Other useful MAAs, such as Gadusol and Palythine also extracted from cyanobacteria, can be incorporated in the bacteria to assess if there is an additive or synergistic UV protective effect along with Shinorine.

Engineering S. epidermidis to Synthesize Sunscreen Molecules

The genes responsible for the synthesis of MAA have been identified in cyanobacteria. The present invention introduces a codon-optimized version of these genes in cis in the nonessential attB locus to confer symbiotic S. epidermidis the ability to produce sufficient quantities of MAAs. Successful chromosomal integration of the genes of interest and biosynthesis of MAAs can be confirmed using the pMAD system, HPLC and LC/MS.

In one embodiment, a method for in vitro safety and efficacy studies is disclosed. A 3D skin model using primary keratinocytes obtained from human foreskin is inoculated with the engineered bacteria and exposed to UV light. The treatment samples are: (i) control S. epidermidis, (ii) recombinant S. epidermidis containing Shinorine gene cluster, (iii) MAAs isolated from engineered S. epidermidis, (iv) MAA extract obtained from marine sources (Helioguard 365), (v) marketed sunscreen molecule viz. octocrylene. The skin samples are exposed to UV radiation at various doses viz. 7, 14, 21, or 35 mJ cm-2. Different UV lamps emitting different wavelengths in the UV-A and UV-B regions are used. Protection from UV light is evaluated by measuring apoptosis, skin proliferation, and selected gene expression. Toxicity is evaluated by checking for key inflammatory mediators and DNA damage.

Regulated Release of Sunscreen Molecules from Engineered S. epidermidis Using a UV-Sensitive Promoter to Allow Photoresponsive Modulation

Bacterial promoters such as recA, lexA, etc., which are part of the robust bacterial SOS response, upregulate gene transcription upon detection of DNA damage (e.g., by UV light). Introducing a relevant UV-sensitive promoter into S. epidermidis allows release of sunscreen molecules commensurate with the intensity of exposure of UV radiation. In one embodiment of the present invention, another stable plasmid (in trans) is introduced in the engineered bacteria consisting of an optimized UV-sensitive promoter and a darkness-inducible host cell lysis sequence derived from the bacteriophage phi X174 that is activated in the absence of UV light. With this system, bacterial lysis can be observed in the dark, yet viability preserved in the presence of UV light and release of sunscreen compounds commensurate with UV exposure. Using HPLC & LC/MS, the amount of MAAs produced can be correlated with the amount of inoculum required and extent of UV exposure. Once both the plasmids responsible for the (i) synthesis of the sunscreen molecule, recA-MAA, and (ii) for the UV radiation-mediated lysis, recA-□XI74E, are inserted into the bacteria, the aforementioned safety and efficacy studies may be performed again to establish the safety and efficacy of the final construct.

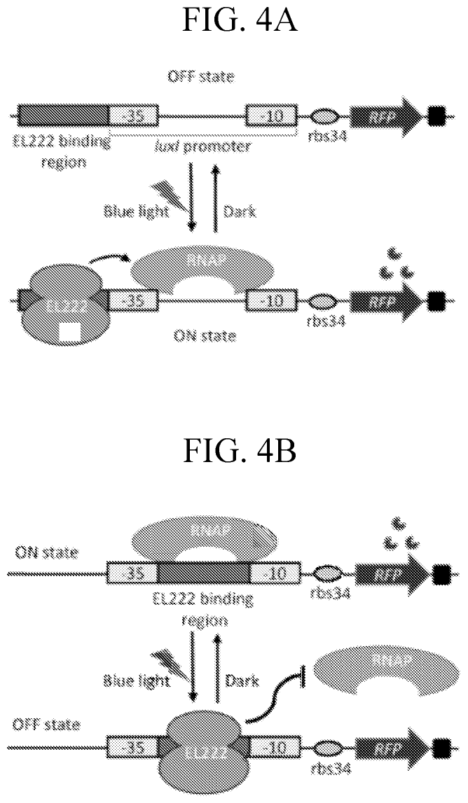

Referring the FIG. 3, HPLC analysis indicates production and release of Shinorine in the bacterial supernatant indicating the successful engineering of S. epidermidis to synthesize Shinorine. A number of optogenetic response elements (ranging from UV-B to far red) are utilized to activate the promoters associated with Shinorine. This allows for a response to UV-B or visible light to activate the production. In this embodiment, optogenetic sensors are used involving single protein systems or combinatorial systems involving PhyB-Pif, CryB-CIB, Cry2, LITEs, LACE, LITEZ, TULIP, EL222, TAEL, LANS, BLITZ, and or UVR8-COP1.

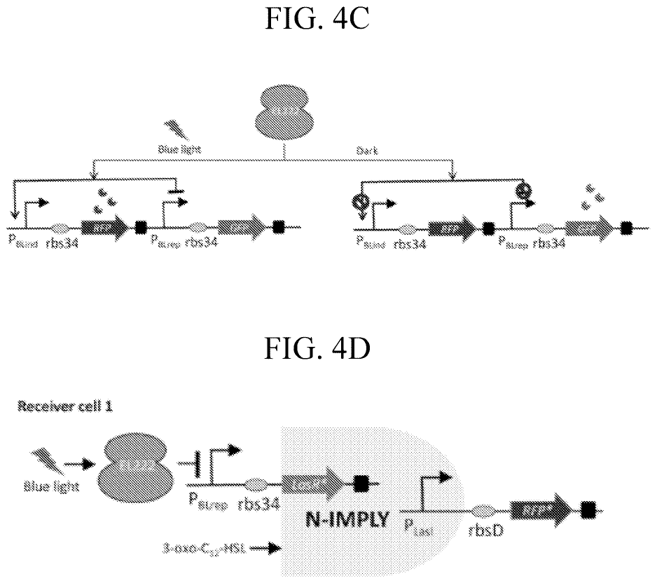

FIGS. 4A-4D are a series of illustrations showing blue light-mediated transcriptional activation and repression of gene expression in bacteria. The illustrations show blue-light inducible EL222 protein from Erythrobacter litoralis. The luxR box is replaced with the EL222 box, which results in specific activation. This specifically describes the Light-Oxygen-Voltage domain and may be an important part of light activated promoters. This demonstrates a novel bidirectional promoter system for Escherichia coli that can be induced or repressed rapidly and reversibly using the blue light dependent DNA-binding protein EL222.

Table 1 shows a list of optogenetic tools for controlling protein-protein interactions and protein oligomerization.

TABLE 1

| | |
|---|---|
| Optogenetic Interaction System, | |
| Chromophore and Color of Activation | Advantages (+) and Disadvantages (−) of the Tools |

Phytochrome

| | |
|---|---|
| −PhyB-PIF3/PIF8 | +bimodal switchable |
| Bilin chromophore | +deep tissue penetration of red/far-red light |
| Activation by red light (660 nm), far-red inactivation (130 nm) | +color tuning possible using different bilin variants |
| | −chromophore not ubiquitously available |

LOV domain

| | |
|---|---|
| FKF1 and GIGANTEA | +ubiquitous chromophore availability |
| −AsLOV2-peptides | +tuned variants with different time constants and affinities |
| −TULIPs | +high dynamic range of improved variants |
| −Magnets | +small size of LOV domain |
| FMN chromophore | −no color tuning |
| Activation by blue light (470 nm) | |

Cryptochrome

| | |
|---|---|
| −Cry2 and CIB(N) | +ubiquitous chromophore availability |
| FAD chromophore | +tuned variants with different time constants and affinities |
| Activation by blue light (470 nm) | +high dynamic range of improved variants |
| | −large protein size |
| | −no color tuning |

UVR8

| | |
|---|---|
| −homodimerization or heterodimerization with Cop1 | +no additional chromophore |
| Intrinsic tryptophan cluster as chromophore | +selective activation when combined with phytochromes |
| Activation by UV-B (280 nm) | +color-tuned variants absorbing UV-C |
| | −UV-light induced photodamage |
| | −irreversible |
| | +increased homodimerization affinity when using UVR8 tandems |

Fluorescent proteins

| | |
|---|---|
| −Dronpa K145N | +bimodal switchable |
| Cys-Trp-Gly as chromophore | +GFP-based: small protein, tunable |
| UV/cyan (variable) | −UV light for activation |
| | −low dynamic range |
| | −only homodimerization |

Table 2 lists a general review of optogenetics tools, from the perspective of mammalian cells. The tools that are microbially sourced are particularly useful for the present invention.

TABLE 2

| | Chromo- | | Tool | Photo | Co- | | Activation | | Intensity | | |
|---|---|---|---|---|---|---|---|---|---|---|---|
| Light | phore | System | type | sensor | factor | TF | wavelength | | (μmol m⁻² s⁻¹)* | | Model |
| RED | PCB | PhyB-Pif | Two- | PhyB | Pif3 | Gal4 | 660 | nm/ | 1 or 40 | | Yeast |
| | | | hybrid | | | DBD- | 750 | nm | | | |
| | | | | | | Gal4 AD | | | | | |
| | | | Two- | PhyB | Pif6 | TetR | 660 | nm/ | 8/80 | | CHO-K1 cells |
| | | | hybrid | | | DBD- | 740 | nm | | | Chicken |
| | | | | | | VP16 AD | | | | | embryos |
| | | | Two- | PhyB | Pif6 | TetR | 660 | nm/ | 20/20 | | NIH/3T3 cells |
| | | | hybrid | | | DBD- | 740 | nm | | | Zebrafish |
| | | | | | | VP16 AD | | | | | |
| BLUE | FDA | CRY2-CIB | Two- | Cry2 | CIB1 | Gal4 | 488 | nm | 25 | μW, | HEK293 cells |
| | | | hybrid | | | DBD- | | | 1.7 | mW, | |
| | | | | | | Gal4 AD | | | 4.5 | mW | |
| | | | Two- | Cry2 | CIB1 | LexA | 474 | nm | 2.5 | mW cm⁻² | S2 cells |
| | | | hybrid | | | DBD- | | | | | Drosophila |
| | | | | | | AD Gal4 | | | | | |
| | | | Hetero- | Cry2 | CIB1 | NA⁻ | Blue | | 42-120 | mmol m⁻² s⁻¹ | Zebrafish |
| | | | dimer | | | | | | | | |
| | | CRY2 | NA | | NA | Gal4 DB | 461 | nm | 7.4 | mW cm⁻² | HEK293 cells |
| | | | | | | VP16AD | | | | | |
| | | LITEs | Hetero- | Cry2 | CIB1 | TALE- | 473 | nm | 5 | mW | Neuro2A cells |
| | | | dimer | | | VP64AD | | | | | |
| | | LACE | Hetero- | Cry2 | CIBN | VP64 | 450 | nm | 48 | lumens | HEK293 cells |
| | | | dimer | | | AD-dCas9 | | | | | |
| | FMN | LightOn | Homo- | VVD | NA⁺ | Gal4 | 460 | nm | 0.84 | Wm⁻² | HEK293 cells |
| | | | dimer | | | (1-65 aa) | | | 90 | mW cm⁻² | Mice |
| | | LITEZ | Two- | GI | FKF1 | ZFP | 450 | nm | 48 | lumens | HEK 293T, |
| | | | hybrid | | | DBD- | | | | | NIH 3T3, |
| | | | | | | VP16AD | | | | | HeLa cells |
| | | TULIP | Two- | LOV-pep | ePDZ | Gal4 | 461 | nm | 5.8 | mW cm⁻² | Yeast |
| | | | hybrid | | | DBD- | | | | | |
| | | | | | | Gal4 AD | | | | | |
| | | EL222 | Dimer | LOV | NA | HTH | 465 | nm | 8 | mW cm⁻² | HEK293 cells |
| | | | | | | DBD- | | | | | Zebrafish |
| | | | | | | VP16AD | | | | | |
| | | TAEL | Dimer | LOV | NA | HTH | 488 | nm | 1.6 | mW cm⁻² | HEK293 cells |
| | | | | | | DBD- | | | | | |
| | | | | | | KalTA4AD | | | | | |
| | | LANS | NLS | asLOV2 | NA | LexA | 455 | nm | 6 | mW cm⁻² | Yeast |
| | | | shuttle | | | DBD- | | | | | |
| | | | | | | Gal4AD | | | | | |
| | | LINuS | NLS | asLOV2 | NA | LexA | 460 | nm | 10 | | Yeast |
| | | | shuttle | | | DBD- | | | | | HEK293 cells |
| | | | | | | VP64AD | | | | | |
| | | LINX | NES | asLOV2 | NA | LexA | 488 | nm | 8 | μS/pixel | HEK293 cells |
| | | | shuttle | | | DBD- | | | | | |
| | | | | | | Gal4AD | | | | | |
| | | LEXY | NES | asLOV2 | NA | LexA | 490 | nm | Not specified | | H1299 cells |
| | | | shuttle | | | DBD- | | | | | |
| | | | | | | VP16AD | | | | | |
| | | BLITZ | Dimer | Cry2/asLOV2 | CIBN/ | TetR- | 473 | nm | 1.7 | mW | HEK293 cells |
| | | | | | NA | VP16AD | | | | | |
| UV-B | NA | UVR8- | Two- | UVR8 | COP1 | Gal4 | 280-375 | nm | 25 | J m⁻² | U2OS cells |
| | | COP1 | hybrid | | | DBD- | 290-310 | nm | 0.7 | mW | |
| | | | | | | NF-κB AD | | | | | |

*Unless specified in other light units; NA, not applicable; NA—light negatively regulate gene expression.

The present invention also encompasses other skin commensal microbiota. Table 3 shows the top 10 abundant bacteria per Byrd et al. Apart from this, other families such as Acinetobacter spp., Bacteroidetes and Proteobacteria can be used.

TABLE 3
Bacteria

| Dry | Moist | Sebaceous | Foot |
|---|---|---|---|
| Propionibacterium acnes | Corynebacterium | Propionibacterium acnes | Corynebacterium |
| | tuberculostearicum | | tuberculostearicum |
| Corynebacterium | Staphylococcus hominis | Staphylococcus epidermidis | Staphylococcus hominis |
| tuberculostearicum | | | |
| Streptococcus mitis | Propionibacterium acnes | Corynebacterium | Staphylococcus warneri |
| | | tuberculostearicum | |
| Streptococcus oralis | Staphylococcus epidermidis | Staphylococcus capitis | Staphylococcus epidermidis |
| Streptococcus pseudopneumoniae | Staphylococcus capitis | Corynebacterium simulans | Staphylococcus capitis |
| Streptococcus sanguinis | Corynebacterium fastidiosum | Streptococcus mitis | Staphylococcus haemolyticus |
| Micrococcus luteus | Corynebacterium afermentans | Staphylococcus hominis | Micrococcus luteus |
| (Gram negative) | | | |
| Staphylococcus epidermidis | Micrococcus luteus | Corynebacterium aurimucosum | Corynebacterium afermentans |
| Staphylococcus capitis | Enterobacter aerosaccus | Corynebacterium kroppenstedtii | Corynebacterium simulans |
| Veillonella parvula | Corynebacterium simulans | Corynebacterium amycolatum | Corynebacterium resistens |
| (Gram negative, anaerobic) | | | |

To facilitate secretion/excretion of the MAA to the external environment, bacteria's natural secretion system is modified to bind and secrete the MAAs that are generated within. This enables the MAA to provide sufficient protection against UV radiation and other photoaging processes. Table 4 highlights the various gram-positive and gram-negative related secretion systems. The table lists genes and transporters related to amino acid production in Corynebacterium glutamicum and Escherichia coli: uptake and excretion systems.

TABLE 4

| | | | |
|---|---|---|---|
| Transporter | Gene(s) | Substrate(s) | Characteristics |

Corynebacterium glutamicum

| | | | |
|---|---|---|---|
| AroP | aroP | L-Tyr, L-Phe | Aromatic amino acids uptake system |
| BrnQ | brnQ | L-Be | Na⁺-coupled uptake system |
| GluABCD | gluABCD | L-Glu | Binding protein-dependent uptake system, expression |
| | | | glucose-repressed |
| Glutamate permease | ? | L-Glu | Uptake active in complex medium |
| LysE | iysE | L-Lys, L-Arg | Exporter, expression regulated by LysG, |
| | | | coinducers L-citrulline and L-histidine |
| Lysl | iysi | L-LyS, L-Ala, L-Val, L-Lev | Low capacity antiporter |
| ThrE | thrE | L-Tlu⁻, L-Ser | Export carrier |

Escherichia coli

| | | | |
|---|---|---|---|
| AroP | aroP | L-Trp, L-Tyr, L-Phe | General uptake system for aromatic amino acids |
| Aspartate/glutamate carrier | ? | L-Asp, L-Glu | Binding protein-dependent uptake system, |
| | | | inhibited by cysteate |
| GltP | gitP | L-Asp, L-Glu | Na⁻F-independent uptake, inhibited by cysteate and |
| | | | 5 hydroxyaspartate |
| GltS | gitS | L-Glu | Na⁻L dependent uptake, inhibited by a |
| | | | methylglutamate |
| Glutamate excretion carrier | | L-Glu | Stringent response-related export |
| LB/1 | INGETAI | L-Len L-Ile, L-Val, L-Ala, L-Thr. L-Hom | Binding-protein-dependent uptake system, expression |
| | | [symbol] | repressed by LRP |
| Orf299 | ydeD | L-Cys and components of the cysteine pathway | Major facilitator protein involved in efflux |
| | | [symbol] | |
| PheP | pheP | L-Phe | High-affinity uptake system specific for phenylalarine |
| RhtA | rhtA | | Confer resistance to high concentrations of |
| | | | homoserine and threonine, putative threonine |
| | | | excretion carriers |
| RhtB | rhtB | | |
| RhtC | rhtC | | |
| SstT | sstT | L-Set, L-Thr | Ne-coupled serine/threonine importer |
| TdcC | tdcC | L-Lea L-Sec. L-Tbr, L-Hom | Importer active under anaerobic conditions |
| Threonine permease | ? | L-Thr, L-Ser | Na⁻L independent uptake system |

[symbol]

indicates data missing or illegible when filed

The present invention is a unique sunscreen technology which not only significantly improves consumer compliance by eliminating the need of frequent reapplications for effective protection, but also circumvents detrimental impact on human health and the environment. Since the strategy of this invention is to deliver the source of the natural sunscreens, the engineered bacteria, as “living skin-protective factories”, it will significantly reduce the cost of the production, unlike current sunscreens that require production, purification and scale-up under good manufacturing practice (GMP) at a pharmaceutical and industrial scale. This platform has the potential to be customizable to different skin and body types-normal, dry, oily—as bacteria other than S. epidermidis, such as those belonging to the Bacteroidetes and Proteobacteria families can be used as templates with unique combinations of promoters and regulatory elements to regulate production, release and activity of synthesized molecules.

In one embodiment, the present invention is a probiotic sunscreen technology that enables continuous and extended release of UV filtering molecules on the skin surface with photo/dark-responsive promoters providing modulated release of these natural sunscreens based on the intensity of UV exposure to mitigate environmental stress.

In another embodiment, the present invention is incorporated in a topical composition (it is applied topically to the skin). In one embodiment, the modified bacteria is present in the topical composition at a concentration of at least 0.1% by weight of the total composition. The topical composition may be in the form of a cream, lotion, emulsion, gel, ointment, liquid or aerosol spray. In another embodiment, the bacteria of the present invention is used in a composition with a traditional sunscreen.

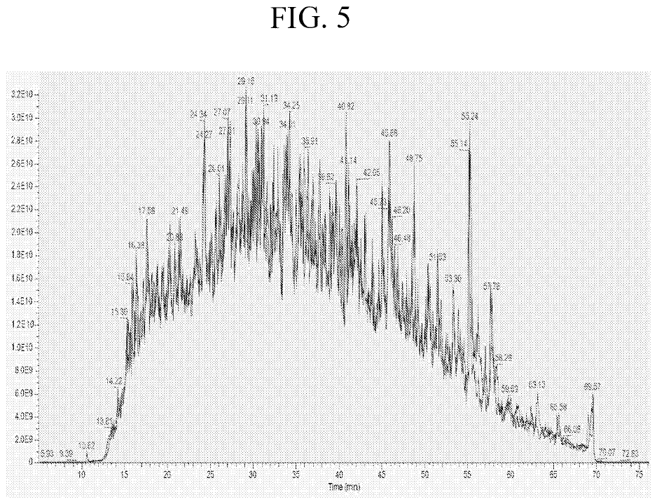

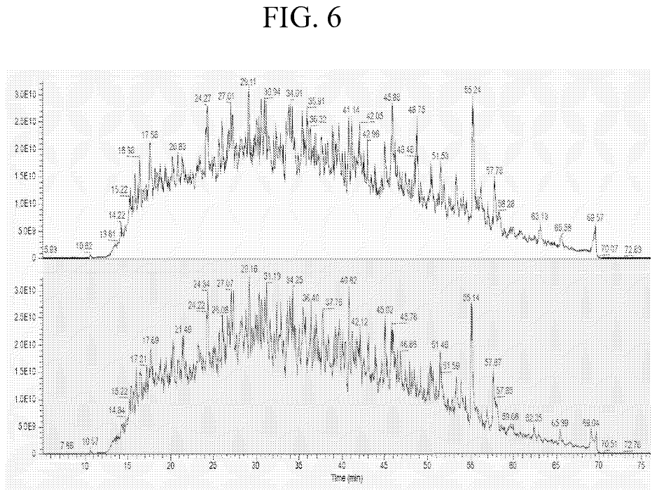

The examples (presented below) show the successful engineering of bacteria containing the genes responsible for MAA production. Transformed colonies submitted for Sanger sequencing gave a positive confirmation for MAA genes. NanoLC-MS/MS data indicated presence of enzymes responsible for production of MAAs. There are four enzymes required to synthesize shinorine and LC-MS/MS chromatogram confirmed their presence (see FIGS. 5 and 6).

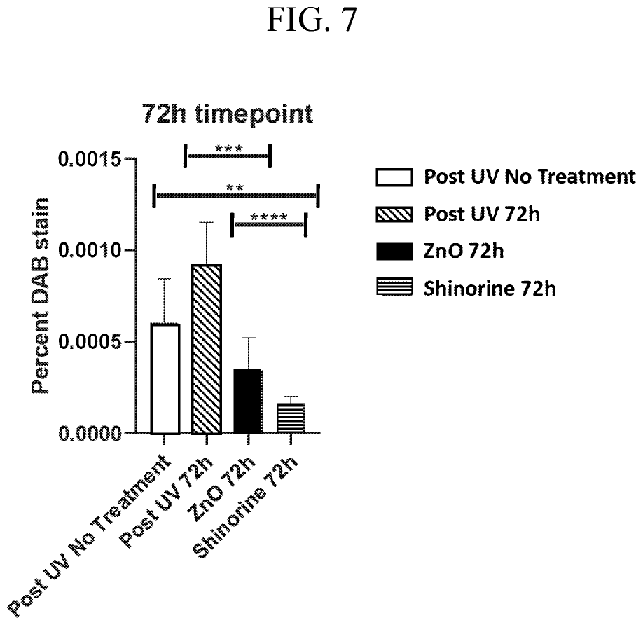

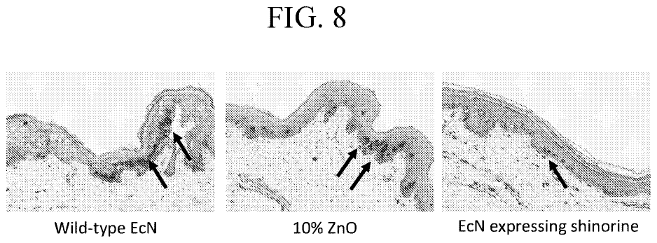

Efficacy studies indicated that shinorine was significantly effective in preventing and/or reducing UV-induced DNA damage compared to untreated group three days post exposure. Furthermore, it was also significantly more effective than a 10% zinc oxide which is a gold standard for sun protection (see FIGS. 7 and 8). This outcome bolsters the photoprotective capacity of shinorine in preventing UV induced skin damage. Evaluating the use of live bacteria on skin tissue also gave encouraging results with IHC images indicating EcN-shinorine's protective effect against UV exposure 24 h post irradiation.

### EXAMPLES

### Example 1—Engineering MAA-Producing Commensal Bacteria

Several studies were conducted on MAAs and the underlying genes in cyanobacteria responsible for their production. Codon-optimized double stranded DNA fragments were amplified using polymerase chain reaction (PCR) and purified using gel electrophoresis. The dsDNA fragments were designed in such a way as to allow blunt end ligation in the pCN48 plasmid using the Smal site. A restriction digest using Smal enzyme was performed to linearize the plasmid, followed by dephosphorylation using calf intestinal phosphatase (CIP) to prevent the plasmid to close onto itself.

The gene of interest is ligated into the plasmid using T4 DNA ligase. Post ligation, chemically competent DH5a cells are transformed and selected using ampicillin. The plasmids from several transformed colonies are isolated and sent for Sanger Sequencing to check and corroborate successful addition of the gene of interest into the plasmid. Bacterial colonies containing the correct plasmid are isolated and the above process is performed again until all four genes of interest are successfully incorporated. The complete plasmid is initially incorporated into E. coli Nissle 1917 (EcN) before being transferred to S. epidermidis.

### Example 2—Validation of MAA Production by the Engineered Bacteria

1% of overnight cultures were inoculated into 100 ml of fresh LB broth supplemented with the appropriate antibiotic and similarly grown at 37° C. and 200 rpm until OD600 reaches 0.9-1.0 (approx. 3.00-6.00 hr.). Cultures were spun at 4° C. for 15 min, 3500×g, and cells were resuspended in 5-10 volumes of the Bacterial cell lysis buffer (Gold Bio), supplemented with DTT and EDTA (5 mM), and Lysozyme (40 mg/ml), DNase (800 U/ml) and RNase (24 U/ml). Following vortexing, and 5 min incubation on ice, suspensions were incubated at 37° C. for 60 min and lysates were centrifuged at 20,000×g, 4° C. for 30 min, and the clear lysate was collected and quantified using BCA assay (Thermo Scientific).

Protein samples were dried in a speed vac and resuspended in TEAB buffer according to standard in-solution digestion protocol. Samples were reduced with TCEP (tris-(2-carboxyethyl) phosphine) and alkylated with MMTS (methyl-methane-thiosulfonate). Samples were digested overnight at 37° C. and reactions were stopped by adding 10% formic acid. These samples were dried and resuspended in 0.1% formic acid. 5 μL (˜1 μg) of each sample was analyzed by NanoLC-MS/MS (Orbitrap Eclipse) and was searched against a combined database consisting of the E. coli Nissle 1917 database accessed from the Biocyc.org website and a database containing the MAA sequences using Proteome discoverer ver 2.4 and the Sequest HT search algorithm using standard LFQ workflow (Thermo scientific).

### Example 3—Establishing the In Vitro Efficacy of Engineered Bacteria

MAAs extracted from the engineered bacteria of the present invention were evaluated. Human skin was obtained from discarded tissue from elective procedures. The skin tissue was cleaned, trimmed, sterilized and plated in an organ culture medium overnight at 33° C. MAA extract or appropriate controls were applied on skin tissues, after which they were exposed to 135 mJ/cm² UV-A and UV-B light. The application of live bacteria on human skin tissue was subsequently tested. Different treatments viz. overnight culture of wild type EcN, 10% zinc oxide solution, or overnight culture of EcN expressing shinorine were applied on human skin explants. Treatments were allowed to equilibrate with skin tissue for 1 hour, after which tissues were exposed to UV radiation. The tissues were then incubated for different durations and subsequently fixed. Cleaved caspase-3 immunohistochemical staining was performed to check for DNA damage. Slides were imaged using Leica Slide Scanner and representative images were captured. The captured images were quantified using ImageJ with a modified, previously published protocol (Crowe and Yue, 2019).

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

SEQUENCES

SEQ ID NO: 1

| | |
|---|---|
| tgattaactt tataggaggt aaaaacatat gagtatcgtc caagcaaagt ttgaagctaa | 60 |
| ggaaacatct tttcatgtag aaggttacga aaagattgag tatgatttgg tgtatgtaga | 120 |
| tggtattttt gaaatccaga attctgcact agcagatgta tatcaaggtt ttggacgatg | 180 |
| cttggcgatt gtagatgcta acgtcagtcg gttgtatggt aatcaaattc aggcatattt | 240 |
| ccagtattat ggtatagaac tgaggctatt tcctattacc attactgaac cagataagac | 300 |
| tattcaaact ttcgagagag ttatagatgt ctttgcagat ttcaaattag tccgcaaaga | 360 |
| accagtatta gtcgtgggtg gcggtttaat tacagatgtt gtcggctttg cttgttctac | 420 |
| atatcgtcgc agcagcaatt acatccgcat tcctactaca ttgattggat taattgatgc | 480 |
| cagtgtagca attaaggtag cagttaatca tcgcaaactg aaaaaccgtt tgggtgctta | 540 |
| tcatgcttct cgcaaagtat ttttagattt ctccttgttg cgtactctcc ctacagacca | 600 |
| agtacgtaac gggatggcgg aattggtaaa aatcgctgta gtagcgcatc aagaagtttt | 660 |
| tgaattgttg gagaagtacg gcgaagaatt actacgtact cattttggca atatagatgc | 720 |
| aactccagag attaaagaaa tagcccatcg tttgacttac aaagctatcc ataagatgtt | 780 |
| ggaattggaa gttcccaacc tgcatgagtt agacctagat agggtgattg cttacggtca | 840 |
| cacttggagt cccaccttgg aacttgcgcc tcgtctaccc atgttccacg gacacgccgt | 900 |
| taatgtagat atggctttct cggcaacgat cgccgcccgt agaggatata ttacaattgc | 960 |
| agaacgcgat cgtattttag gattaatgag tcgcgttggt ctatccctcg accatcccat | 1020 |
| gttggatata gatattttgt ggcgtggtac tgaatctatc acattaactc gtgatggttt | 1080 |
| gttaagagct gctatgccaa aacccattgg tgattgtgtc ttcgtcaatg acctgacaag | 1140 |
| agaagaatta gcagccgcat tagctgacca caaagaactt tgtaccagtt atccccgtgg | 1200 |
| tggtgaaggt gtggatgtgt atcccgttta tcaaaaagaa ttaatcggga gtgttaaata | 1260 |
| a | 1261 |

SEQ ID NO: 2

| | |
|---|---|
| tgattaactt tataaggagg aaaaacatat gctttctggt catatcgaag gacaaacctt | 60 |
| aaagatgttt gttcacatga ccaaagctaa aaaagtctta gaaattggga tgtttaccgg | 120 |
| ttattcggcg ctggcgatgg cggaagcatt accagaggat ggactgcttg tggcttgtga | 180 |
| agttgaccct tacgcggcgg aaattggaca gaaagccttt caacaatctc cccacggtgg | 240 |
| aaagattcgt gtggaattgg atgcagcctt agcaactctt gataagttag cagaagctgg | 300 |
| ggagtctttt gacttggtat ttatcgacgc agataaaaaa gagtatgtag cctattttca | 360 |
| caagttgcta ggtagcagtt tgttagcacc agatggcttt atttgtgtag ataacacctt | 420 |
| attacaaggg gaagtttatc taccagcaga ggaacgtagc gtcaatggtg aagcgatcgc | 480 |
| gcaatttaat catacagtag ctatagaccc ccgtgtagaa caggttttgt tgccgttgcg | 540 |
| agatggttta acaattatcc gcagaataca accttaa | 577 |

SEQ ID NO: 3

| | |
|---|---|
| tgattaactt tataaggagg aaaaacatat ggcacaatcc cttccccttt cttccgcacc | 60 |
| tgctacaccg tctcttcctt cccagacgaa aatagccgca attatccaaa atatctgcac | 120 |
| tttggctttg ttattactag cattgcccat taatgccacc attgttttta tatccttgtt | 180 |
| agtcttccga ccgcaaaagg tcaaagcagc aaacccccaa accattctta tcagtggcgg | 240 |
| taagatgacc aaagctttac aactagcaag gtcattccac gcggctggac atagagttgt | 300 |
| cttggtggaa acccataaat actggttgac tggtcatcgt ttttcccaag cagtggataa | 360 |
| gttttacaca gtccccgcac cccaggacaa tccccaagct tacattcagg ctttggtaga | 420 |
| tatcgtcaaa caagaaaaca tcgatgttta tattcccgtc accagtccag tgggtagcta | 480 |
| ctacgactca ttagccaaac cagagttatc ccattattgc gaagtgtttc actttgacgc | 540 |
| agatattacc caaatgttgg atgataaatt tgcgttgaca caaaaagcgc gatcgcttgg | 600 |
| tttatcagta cccaaatcct ttaaaattac ctcaccagaa caagtcatca acttcgattt | 660 |
| ttctggagag acacgtaaat acatcctcaa aagcattccc tacgactcag tgcggcggtt | 720 |
| ggacttaacc aaactcccct gtgctactcc agaggaaaca gcagcattcg tcagaagttt | 780 |
| gccaattact cccgaaaaac cgtggattat gcaggaattt atccccggta aggaattctg | 840 |
| cacccatagc accgttcgga atggggaact cagactgcat tgctgttgcg aatcttcagc | 900 |
| cttccaagtt aattatgaga atgtaaataa cccgcaaatt accgaatggg tacagcattt | 960 |
| tgtcaaggaa ctgaaactga caggacagat ttcctttgac tttatccaag ccgaagacgg | 1020 |
| aacagtttac gccatcgagt gtaacccccg cacacattca gcaattacca cattttacga | 1080 |
| ccacccccag gtagcagaag cgtacttgag tcaagcaccg acgactgaaa ccatacaacc | 1140 |
| actaacgaca agcaagccta cctattggac ttatcacgaa gtttggcgtt taactggtat | 1200 |
| ccgttctttc acccagttgc aaagatggct ggggaatatt tggcgcggga ctgatgcgat | 1260 |
| ttatcagcca gatgacccct taccgttttt gatggtacat cattggcaaa ttcccctact | 1320 |
| gttattgaat aatttgcgtc gtcttaaagg ttggacgcgg atagatttca atattgggaa | 1380 |
| gttggtggaa ttggggggag attag | 1405 |

SEQ ID NO: 4

| | |
|---|---|
| atgcagacta tagattttaa tattcgtaag ttacttgtag agtggaacgc gacccacaga | 60 |
| gattatgatc tttcccagag tttacatgaa ctaattgtag ctcaagtaga acgaacacct | 120 |
| gaggcgatcg ctgtcacctt tgacaagcaa caactaactt atcaagaact aaatcataaa | 180 |
| gcaaaccagc taggacatta tttacaaaca ttaggagtcc agccagaaac cctggtaggc | 240 |
| gtttgtttag aacgttcctt agaaatggtt atctgtcttt taggaatcct caaagctggg | 300 |
| ggtgcttatg ttcctattga ccctgaatat cctcaagaac gcatagctta tatgctagaa | 360 |
| gattctcagg tgaaggtact actaactcaa gaaaaattac tcaatcaaat tccccaccat | 420 |
| caagcacaaa ctatctgtgt agatagggaa tgggagaaaa tttccacaca agctaatacc | 480 |
| aatcccaaaa gtaatataaa aacggataat cttgcttatg taatttacac ctctggttcc | 540 |
| actggtaaac caaaaggtgc aatgaacacc cacaaaggta tctgtaatcg cttattgtgg | 600 |
| atgcaggaag cttatcaaat cgattccaca gatagcattt tacaaaaaac cccctttagt | 660 |
| tttgatgttt ccgtttggga gttcttttgg actttattaa ctggcgcacg tttggtaata | 720 |
| gccaaaccag gcggacataa agatagtgct tacctcatcg atttaattac tcaagaacaa | 780 |
| atcactacgt tgcattttgt cccctcaatg ctgcaagtgt ttttacaaaa tcgccatgta | 840 |
| agcaaatgca gctctctaaa aagagttatt tgtagcggtg aagctttatc tatagattta | 900 |
| caaaatagat ttttccagca tttgcaatgt gaattacata acctctatgg cccgacagaa | 960 |
| gcagcaattg atgtcacatt ttggcaatgt agaaaagata gtaatttaaa gagtgtacct | 1020 |
| attggtcgtc ccattgctaa tactcaaatt tatattcttg atgccgattt acaaccagta | 1080 |
| aatattggtg tcactggtga aatttatatt ggtggtgtag gggttgctcg tggttatttg | 1140 |
| aataaagaag aattgaccaa agaaaaattt attattaatc cctttcccaa ttctgagttt | 1200 |
| aagcgacttt ataaaacagg tgatttagct cgttatttac ccgatggaaa tattgaatat | 1260 |
| cttggtagaa cagattatca agtaaaaatt cggggttata gaattgaaat tggcgagatt | 1320 |
| gaaaatgttt tatcttcaca cccacaagtc agagaagctg tagtcatagc gcgggatgat | 1380 |
| aacgctcaag aaaaacaaat catcgcttat attacctata actccatcaa acctcagctt | 1440 |
| gataatctgc gtgatttcct aaaagcaagg ctacctgatt ttatgattcc agccgctttt | 1500 |
| gtgatgctgg agcatcttcc tttaactccc agtggtaaag tagaccgtaa ggcattacct | 1560 |
| aagcctgatt tatttaatta tagtgaacat aattcctatg tagcgcctcg gaatgaagtt | 1620 |
| gaagaaaaat tagtacaaat ctggtcgaat attctgcatt tacctaaagt aggtgtgaca | 1680 |
| gaaaactttt tcgctattgg tggtaattcc ctcaaagctc tacatttaat ttctcaaatt | 1740 |
| gaagagttat ttgctaaaga gatatcctta gcaacacttt taacaaatcc agtaattgca | 1800 |
| gatttagcca aggttattca agcaaacaac caaatccata attcacccct agttccaatt | 1860 |
| caaccacaag gtaagcagca gcctttcttt tgtatacatc ctgctggtgg tcatgtttta | 1920 |
| tgctatttta aactcgcaca atatatagga actgaccaac cattttatgg cttacaagct | 1980 |
| caaggatttt atggagatga agcacccttg acgcgagttg aagatatggc tagtctctac | 2040 |
| gtcaaaacta ttagagaatt tcaaccccaa gggccttatc gtgtcggggg gtggtcattt | 2100 |
| ggtggagtcg tagcttatga agtagcacag cagttacata gacaaggaca agaagtatct | 2160 |
| ttactagcaa tattagattc ttacgtaccg attctgctgg ataaacaaaa acccattgat | 2220 |
| gacgtttatt tagttggtgt tctctccaga gtttttggcg gtatgtttgg tcaagataat | 2280 |
| ctagtcacac ctgaagaaat agaaaattta actgtagaag aaaaaattaa ttacatcatt | 2340 |
| gataaagcac ggagcgctag aatattcccg cctggtgtag aacgtcaaaa taatcgccgt | 2400 |
| attcttgatg ttttggtggg aactttaaaa gcaacttatt cctatataag acaaccatat | 2460 |
| ccaggaaaag tcactgtatt tcgagccagg gaaaaacata ttatggctcc tgacccgacc | 2520 |
| ttagtttggg tagaattatt ttctgtaatg gcggctcaag aaattaagat tattgatgtc | 2580 |
| cctggaaacc attattcgtt tgttctagaa ccccatgtac aggttttagc acagcgttta | 2640 |
| caagattgtc tggaaaataa ttcatgactc ga | 2672 |

SEQ ID NO: 5

| | |
|---|---|
| cctttgcgaa agagttaata agttaacaga agatgaacca aaactaaatg gtttagcagg | 60 |
| aaacttagat aaaaaaatga atccagaatt atattcagaa caggaacagc aacaagaaca | 120 |
| acaaaagaat caaaaacgag atagaggtat gcacttatag aacatgcatt tatgccgaga | 180 |
| aaacttattg gttggaatgg gctatgtgtt agctaacttg ttagcgagtt ggttggactt | 240 |
| gaattgggat taatcccaag aaagtaccaa ctcaacaaca cataaagccc tgtaggttcc | 300 |
| gaccaataag gaaattggaa taaagcaata aaaggagttg aagaaatgaa attcagagaa | 360 |
| gcctttgaga attttataac aagtaagtat gtacttggtg ttttagtagt tttaactgtt | 420 |
| taccagataa tacaaatgct taaataaaaa aagacttgat ctgattagac caaatctttt | 480 |
| gatagtgtta tattaataac aaaataaaaa ggagtcgctc acgccctacc aaagtttgtg | 540 |
| aacgacatca ttcaaagaaa aaaacactga gttgttttta taatcttgta tatttagata | 600 |
| ttaaacgata tttaaatata catcaagata tatatttggg tgagcgatta cttaaacgaa | 660 |
| attgagatta aggagtcgat tttttatgta taaaaacaat catgcaaatc attcaaatca | 720 |
| tttggaaaat cacgatttag acaatttttc taaaaccggc tactctaata gccggttgga | 780 |
| cgcacatact gtgtgcatat ctgatccaaa attaagtttt gatgcaatga cgatcgttgg | 840 |
| aaatctcaac cgagacaacg ctcaagccct ttctaaattt atgagtgtag agccccaaat | 900 |
| aagactttgg gatattcttc aaacaaagtt taaagctaaa gcacttcaag aaaaagttta | 960 |
| tattgaatat gacaaagtga aagcagatag ttgggataga cgtaatatgc gtattgaatt | 1020 |
| taatccaaac aaacttacac gagatgaaat gatttggtta aaacaaaata taataagcta | 1080 |
| catggaagat gacggtttta caagattaga tttagccttt gattttgaag atgatttgag | 1140 |
| tgactactat gcaatgtctg ataaagcagt taagaaaact attttttatg gtcgtaatgg | 1200 |
| taagccagaa acaaaatatt ttggcgtgag agatagtaat agatttatta gaatttataa | 1260 |
| taaaaagcaa gaacgtaaag ataatgcaga tgctgaagtt atgtctgaac atttatggcg | 1320 |
| tgtagaaatc gaacttaaaa gagatatggt ggattactgg aatgattgct ttagtgattt | 1380 |
| acatatcttg caaccagatt ggaaaactat ccaacgcact gcggatagag caatagtttt | 1440 |
| tatgttattg agtgatgaag aagaatgggg aaagcttcac agaaattcta gaacaaaata | 1500 |
| taagaatttg ataaaagaaa tttcgccagt cgatttaacg gacttaatga aatcgacttt | 1560 |
| aaaagcgaac gaaaaacaat tgcaaaaaca aatcgatttt tggcaacatg aatttaaatt | 1620 |
| ttggaaatag tgtacatatt aatattactg aacaaaaatg atatatttaa actattctaa | 1680 |
| tttaggagga tttttttatg aagtgtctat ttaaaaattt ggggaattta tatgaggtga | 1740 |
| aagaataatt tacccctata aactttagtc acctcaagta aagaggtaaa attgtttagt | 1800 |
| ttatataaaa aatttaaagg tttgttttat agcgttttat tttggctttg tattctttca | 1860 |
| ttttttagtg tattaaatga aatggtttta aatgtttctt tacctgatat tgcaaatcat | 1920 |
| tttaatacta ctcctggaat tacaaactgg gtaaacactg catatatgtt aactttttcg | 1980 |
| ataggaacag cagtatatgg aaaattatct gattatataa atataaaaaa attgttaatt | 2040 |
| attggtatta gtttgagctg tcttggttca ttgattgctt ttattgggcc cacctaggaa | 2100 |
| ttgaatgaga catgctacac ctccggataa taaatatata taaacgtata tagatttcat | 2160 |
| aaagtctaac acactagact tatttacttc gtaattaagt cgttaaaccg tgtgctctac | 2220 |
| gaccaaaact ataaaacctt taagaacttt ctttttttac aagaaaaaag aaattagata | 2280 |
| aatctctcat atcttttatt caataatcgc atccgattgc agtataaatt taacgatcac | 2340 |
| tcatcatgtt catatttatc agagctcgtg ctataattat actaatttta taaggaggaa | 2400 |
| aaaatatggg catttttagt atttttgtaa tcagcacagt tcattatcaa ccaaacaaaa | 2460 |
| aataagtggt tataatgaat cgttaataag caaaattcat ataaccaaat taaagagggt | 2520 |
| tataatgaac gagaaaaata taaaacacag tcaaaacttt attacttcaa aacataatat | 2580 |
| agataaaata atgacaaata taagattaaa tgaacatgat aatatctttg aaatcggctc | 2640 |
| aggaaaaggc cattttaccc ttgaattagt aaagaggtgt aatttcgtaa ctgccattga | 2700 |
| aatagaccat aaattatgca aaactacaga aaataaactt gttgatcacg ataatttcca | 2760 |
| agttttaaac aaggatatat tgcagtttaa atttcctaaa aaccaatcct ataaaatata | 2820 |
| tggtaatata ccttataaca taagtacgga tataatacgc aaaattgttt ttgatagtat | 2880 |
| agctaatgag atttatttaa tcgtggaata cgggtttgct aaaagattat taaatacaaa | 2940 |
| acgctcattg gcattacttt taatggcaga agttgatatt tctatattaa gtatggttcc | 3000 |
| aagagaatat tttcatccta aacctaaagt gaatagctca cttatcagat taagtagaaa | 3060 |
| aaaatcaaga atatcacaca aagataaaca aaagtataat tatttcgtta tgaaatgggt | 3120 |
| taacaaagaa tacaagaaaa tatttacaaa aaatcaattt aacaattcct taaaacatgc | 3180 |
| aggaattgac gatttaaaca atattagctt tgaacaattc ttatctcttt tcaatagcta | 3240 |
| taaattattt aataagtaag ttaagggatg cataaactgc atcccttaac ttgtttttcg | 3300 |
| tgtgcctatt ttttgtgaat cgattatgtc ttttgcgcag tcggcttaaa ccagttttcc | 3360 |
| gcggcgctcg agcggccgca tagttaagcc agccccgaca cccgccaaca cccgctgacg | 3420 |
| cgccctgacg ggcttgtctg ctcccggcat ccgcttacag acaagctgtg accgtctccg | 3480 |
| ggagctgcat gtgtcagagg ttttcaccgt catcaccgaa acgcgcgaga cgaaagggcc | 3540 |
| tcgtgatacg cctattttta taggttaatg tcatgataat aatggtttct tagacgtcag | 3600 |
| gtggcacttt tcggggaaat gtgcgcggaa cccctatttg tttatttttc taaatacatt | 3660 |
| caaatatgta tccgctcatg agacaataac cctgataaat gcttcaataa tattgaaaaa | 3720 |
| ggaagagtat gagtattcaa catttccgtg tcgcccttat tccctttttt gcggcatttt | 3780 |
| gccttcctgt ttttgctcac ccagaaacgc tggtgaaagt aaaagatgct gaagatcagt | 3840 |
| tgggtgcacg agtgggttac atcgaactgg atctcaacag cggtaagatc cttgagagtt | 3900 |
| ttcgccccga agaacgtttt ccaatgatga gcacttttaa agttctgcta tgtggcgcgg | 3960 |
| tattatcccg tattgacgcc gggcaagagc aactcggtcg ccgcatacac tattctcaga | 4020 |
| atgacttggt tgagtactca ccagtcacag aaaagcatct tacggatggc atgacagtaa | 4080 |
| gagaattatg cagtgctgcc ataaccatga gtgataacac tgcggccaac ttacttctga | 4140 |
| caacgatcgg aggaccgaag gagctaaccg cttttttgca caacatgggg gatcatgtaa | 4200 |
| ctcgccttga tcgttgggaa ccggagctga atgaagccat accaaacgac gagcgtgaca | 4260 |
| ccacgatgcc tgtagcaatg gcaacaacgt tgcgcaaact attaactggc gaactactta | 4320 |
| ctctagcttc ccggcaacaa ttaatagact ggatggaggc ggataaagtt gcaggaccac | 4380 |
| ttctgcgctc ggcccttccg gctggctggt ttattgctga taaatctgga gccggtgagc | 4440 |
| gtgggtctcg cggtatcatt gcagcactgg ggccagatgg taagccctcc cgtatcgtag | 4500 |
| ttatctacac gacggggagt caggcaacta tggatgaacg aaatagacag atcgctgaga | 4560 |
| taggtgcctc actgattaag cattggtaac tgtcagacca agtttactca tatatacttt | 4620 |
| agattgattt aaaacttcat ttttaattta aaaggatcta ggtgaagatc ctttttgata | 4680 |
| atctcatgac caaaatccct taacgtgagt tttcgttcca ctgagcgtca gaccccgtag | 4740 |
| aaaagatcaa aggatcttct tgagatcctt tttttctgcg cgtaatctgc tgcttgcaaa | 4800 |
| caaaaaaacc accgctacca gcggtggttt gtttgccgga tcaagagcta ccaactcttt | 4860 |
| ttccgaaggt aactggcttc agcagagcgc agataccaaa tactgttctt ctagtgtagc | 4920 |
| cgtagttagg ccaccacttc aagaactctg tagcaccgcc tacatacctc gctctgctaa | 4980 |
| tcctgttacc agtggctgct gccagtggcg ataagtcgtg tcttaccggg ttggactcaa | 5040 |
| gacgatagtt accggataag gcgcagcggt cgggctgaac ggggggttcg tgcacacagc | 5100 |
| ccagcttgga gcgaacgacc tacaccgaac tgagatacct acagcgtgag ctatgagaaa | 5160 |
| gcgccacgct tcccgaaggg agaaaggcgg acaggtatcc ggtaagcggc agggtcggaa | 5220 |
| caggagagcg cacgagggag cttccagggg gaaacgcctg gtatctttat agtcctgtcg | 5280 |
| ggtttcgcca cctctgactt gagcgtcgat ttttgtgatg ctcgtcaggg gggcggagcc | 5340 |
| tatggaaaaa cgccagcaac gcggcctttt tacggttcct ggccttttgc tggccttttg | 5400 |
| ctcacatgtt ctttcctgcg ttatcccctg attctgtgga taaccgtatt accgcctttg | 5460 |
| agtgagctgg cggccgctgc atgcctgcag gtcgactcta gaggatcccc cccctgatat | 5520 |
| ttttgactaa accaaatgct aacccagaaa tacaatcact gtgtctaatg aataatttgt | 5580 |
| tttataaaca cttttttgtt tacttctcat ttttaattag ttataattaa ctaaataata | 5640 |
| gagcattaaa tatatttaat aaaacttatt taatgcaaaa ttatgactaa catatctata | 5700 |
| ataaataaag attagatatc aatatattat cgggcaaatg tatcgagcaa gatgcatcgg | 5760 |
| atcgatccag gaggtatacc tgattaactt tataaggagg aaaaacatat gagtatcgtt | 5820 |
| caagcgaaat ttgaagcaaa agaaacgagt ttccatgtgg aaggatatga gaagatcgag | 5880 |
| tacgatttgg tgtatgttga cggcattttt gagatacaaa attctgcgtt ggcggatgta | 5940 |
| tatcagggat ttggtagatg cttggcgata gttgatgcga atgtgagtcg tttgtatggt | 6000 |
| aatcagatcc aggcgtattt ccaatactat ggtattgagt tacgtttgtt tcctataacg | 6060 |
| attacggagc ctgataagac gattcagact ttcgagcgag ttattgacgt ctttgctgac | 6120 |
| ttcaagttag tcagaaaaga accagtcttg gtagtgggtg gtggattaat cacagacgta | 6180 |
| gtaggcttcg cgtgcagtac ttacagaaga tcttctaact acatccgaat accgacaacg | 6240 |
| ttaataggtt tgattgatgc tagtgttgct atcaaggtgg cagtaaatca cagaaaatta | 6300 |
| aagaaccgat tgggagctta ccacgcatca cgaaaagtct tcttagactt ttctttgtta | 6360 |
| cgaacattgc cgactgatca agtcagaaac ggaatggctg aattagtaaa gatcgcggtc | 6420 |
| gtggcacatc aggaggtgtt cgagttgtta gagaagtacg gagaagagtt attacgaacg | 6480 |
| catttcggta atatagacgc tactcctgaa attaaggaga tcgcgcaccg attgacatac | 6540 |
| aaagctattc ataagatgtt agagttggag gttcctaact tacacgagtt ggacttagac | 6600 |
| cgtgtaatag cttatggtca tacgtggagt ccgacattag aattggctcc tcgtttgcct | 6660 |
| atgttccacg gacacgcggt caacgtcgat atggcattca gtgcgacgat tgctgcacga | 6720 |
| cgaggttaca ttacgattgc tgagcgtgat agaatcttag gattaatgag tcgagtcggt | 6780 |
| ttaagtttgg accaccctat gttggatatt gacatcttat ggcgaggtac agaatctatc | 6840 |
| actttaactc gagacggatt attgcgtgct gcaatgccga agcctatagg cgactgtgta | 6900 |
| ttcgtcaatg atttgactcg agaagaattg gcagcagcgt tagcggacca caaagaatta | 6960 |
| tgtacgagtt atccgcgtgg tggtgaaggc gtcgatgtct atccagtata tcaaaaagag | 7020 |
| ttaatcggaa gtgtaaagta acccccctga ttaactttat aaggaggaaa aacatatgtt | 7080 |
| atcaggacat attgaaggac aaactttaaa gatgtttgtt catatgacga aggctaagaa | 7140 |
| ggtattagaa attggaatgt tcacaggcta ctctgcgtta gcgatggcag aggcgttacc | 7200 |
| agaggatggt ttgttagtcg cgtgcgaagt tgatccttac gcagcggaga taggtcaaaa | 7260 |
| ggcattccag caatcaccac acggcggcaa gatacgagtt gagttggacg cagctttagc | 7320 |
| gactttggac aaattagcag aagcaggaga gagttttgat ttggttttca tagacgctga | 7380 |
| caagaaggaa tacgtggctt attttcataa gttgttggga agttcattat tagctccaga | 7440 |
| cggtttcata tgcgttgaca acactttgtt gcaaggagaa gtatatttac cggcggagga | 7500 |
| gcgatcagtg aatggtgagg ctatcgcaca gttcaatcat actgtcgcaa ttgatccgcg | 7560 |
| agtcgagcaa gtgttattgc cgttgagaga tggtttaact attattcgtc gtattcagcc | 7620 |
| ataacccccc tgattaactt tataaggagg aaaaacatat ggctcaatct ttaccattat | 7680 |
| cttctgcacc agcgactccg tcattgccat cacagactaa gattgctgcg atcatccaga | 7740 |
| atatctgtac gttggcgttg ttgttgttgg ctttgccaat taacgctacg atagtgttta | 7800 |
| tcagtttgtt agtatttcga ccacagaagg tgaaggctgc taacccacaa acaatattaa | 7860 |
| tctcaggtgg aaagatgact aaagcattgc agttagcacg atctttccat gcagctggtc | 7920 |
| atagagttgt tttggtggaa acgcataagt attggttgac tggtcacaga ttctcacagg | 7980 |
| cagtagacaa attttatact gtccctgcac cgcaagacaa tccgcaagcg tatattcaag | 8040 |
| ctttggtaga cattgtcaaa caggaaaata tagacgtcta tattccggtg acatctccgg | 8100 |
| tcggctcata ttacgattca ttggcgaagc cggaattgtc tcactattgt gaagtgtttc | 8160 |
| acttcgatgc agacataaca caaatgttag acgacaaatt tgctttaact caaaaagcac | 8220 |
| gatctttggg cttgtcagtc ccgaagtctt tcaaaataac ttctccggag caagtaatta | 8280 |
| acttcgactt ctcaggagaa actagaaaat acatcttaaa aagtattcca tacgacagtg | 8340 |
| tacgtcgttt agatttgaca aagttacctt gtgctacgcc ggaagagaca gcagctttcg | 8400 |
| ttagaagttt gccgatcacg ccagagaagc cgtggataat gcaggaattt ataccaggca | 8460 |
| aggaattttg tacacactca actgtcagaa acggagaatt acgtttgcac tgctgttgtg | 8520 |
| agtcatcagc tttccaagtc aactatgaga acgtaaacaa tccgcagata acagaatggg | 8580 |
| tccaacattt tgtcaaagag ttaaagttga ctggacaaat ctctttcgat ttcatacaag | 8640 |
| ctgaagatgg aacagtatac gctattgagt gtaaccctcg aactcattca gcgataacta | 8700 |
| ctttctatga ccatccgcaa gttgcagaag cttacttgtc acaggctcca acaacggaga | 8760 |
| caattcaacc attaacaaca tctaaaccaa cttattggac atatcatgaa gtgtggagat | 8820 |
| taacgggtat ccgaagtttc actcagttgc aacgatggtt gggcaacatt tggcgtggca | 8880 |
| ctgatgcgat ttaccaaccg gacgacccgt tacctttctt gatggttcat cattggcaaa | 8940 |
| ttccgttgtt attgttgaat aatttgcgac gattaaaggg ttggacacgt attgatttca | 9000 |
| acattggaaa attggtcgaa ttaggaggtg actagccccc ctgattaact ttataaggag | 9060 |
| gaaaaacata tgcagacgat tgattttaat atccgaaagt tgttagtgga atggaacgcg | 9120 |
| acacatcgtg attacgactt gtcacagtct ttgcacgaat tgatagttgc acaagttgaa | 9180 |
| agaactcctg aagctattgc tgttacgttc gacaagcagc aattaacgta tcaggaatta | 9240 |
| aatcataaag cgaaccagtt gggacactac ttacaaacgt taggtgtcca accggagacg | 9300 |
| ttagtcggtg tctgtttgga acgtagtttg gagatggtca tttgtttatt aggaatattg | 9360 |
| aaggcgggcg gtgcttatgt ccctatcgac ccggaatatc ctcaggaacg tatagcttac | 9420 |
| atgttagaag actctcaggt gaaggttttg ttgactcaag aaaaattatt aaatcagatc | 9480 |
| ccgcaccatc aggcacaaac aatatgtgtt gatagagaat gggagaaaat ctctacacaa | 9540 |
| gcgaatacaa atccgaaatc aaatattaag acggataact tggcatacgt catttacact | 9600 |
| tctggtagta caggaaaacc aaaaggtgcg atgaacacgc ataaaggcat atgtaatcga | 9660 |
| ttattgtgga tgcaagaggc ttatcagatc gatagtacgg acagtatctt gcaaaaaacg | 9720 |
| ccgttctctt ttgatgtttc agtctgggag tttttctgga cattattgac gggagcgcga | 9780 |
| ttggttatcg ctaagccagg aggccacaaa gacagtgcat atttgataga tttgataaca | 9840 |
| caggagcaaa ttacaacttt acactttgtg ccgtcaatgt tacaagtctt cttacagaac | 9900 |
| cgtcacgtaa gtaagtgtag ttctttaaag cgagtcattt gctcaggaga ggcgttatca | 9960 |
| attgatttac agaatagatt tttccaacac ttgcagtgtg agttgcacaa cttatatggt | 10020 |
| ccaacagaag ctgctattga cgtaactttt tggcaatgta gaaaggattc taatttaaaa | 10080 |
| tctgtcccga taggtagacc tatagcgaat acacaaatat atatcttgga cgctgattta | 10140 |
| cagccggtta acataggcgt aacgggagag atttatattg gaggtgtagg cgtggctcgt | 10200 |
| ggatatttga ataaagagga gttaactaaa gaaaaattca tcatcaatcc tttcccaaac | 10260 |
| tctgaattca aacgtttgta taaaacgggc gatttagcgc gttatttacc agacggcaac | 10320 |
| atagaatatt tgggcagaac agattatcaa gtgaaaatac gaggctatag aatagaaata | 10380 |
| ggcgaaatag agaacgtctt gtctagtcat ccacaggtga gagaagctgt cgtaatagct | 10440 |
| agagatgata acgcgcaaga gaaacagatc atcgcttaca tcacatataa tagtatcaag | 10500 |
| ccgcaattgg acaacttacg agacttcttg aaggcacgtt tgccggattt tatgattcct | 10560 |
| gcagcttttg tgatgttgga acacttaccg ttgactccaa gtggcaaagt agatcgaaag | 10620 |
| gcattgccta agccagattt attcaattac tcagaacata attcttatgt tgcaccgcgt | 10680 |
| aatgaagtag aggaaaaatt ggtgcagata tggtctaaca ttttacattt acctaaagta | 10740 |
| ggcgttactg aaaacttttt cgcgatcggt ggtaatagtt tgaaggcgtt acacttaatt | 10800 |
| agtcagattg aggaattatt cgcaaaggag attagtttgg cgacgttgtt gacaaatcca | 10860 |
| gttattgcgg acttagctaa agttattcaa gcgaataacc aaatccacaa ttcaccatta | 10920 |
| gtgccgatcc aaccgcaggg caagcagcaa ccttttttct gtatccatcc ggcgggtgga | 10980 |
| catgtattat gttacttcaa gttggcgcaa tacataggaa cggaccaacc attttacggc | 11040 |
| ttgcaggctc aaggttttta cggagatgaa gcgccattga cacgtgttga ggatatggca | 11100 |
| tctttgtacg tgaaaacgat acgagagttc caacctcaag gcccgtaccg agtcggtggc | 11160 |
| tggtctttcg gcggcgtagt ggcgtacgag gtcgcgcaac aattgcatcg acaaggacaa | 11220 |
| gaggtctcat tgttagcaat tttagatagt tatgtgccaa tcttattgga taaacaaaag | 11280 |
| ccaatcgatg atgtgtactt ggtgggagtc ttgtctcgtg tcttcggtgg aatgttcggt | 11340 |
| caagacaatt tagtaacacc tgaagaaatc gaaaatttga cggtagaaga gaaaatcaat | 11400 |
| tacatcattg ataaagctag atcagcacgt atttttcctc cgggagtaga acgacagaac | 11460 |
| aatcgtcgta ttttggatgt cttagtgggc actttgaaag caacttatag ttacattcga | 11520 |
| cagccttatc cgggcaaggt cacagttttc cgtgcgcgtg aaaaacatat aatggcgccg | 11580 |
| gacccaactt tggtctgggt tgaattattt tcagttatgg cggcgcagga aataaagatc | 11640 |
| atagacgtac cgggcaatca ctactctttt gtcttagaac cacatgtaca agtattggct | 11700 |
| cagagattac aggactgctt agaaaataac tcatgacccg ggtaccgagc tcgaattcag | 11760 |
| gcgcgcctat tctaaatgca taataaatac tgataacatc ttatattttg tattatattt | 11820 |
| tgtattatcg ttgacatgta taattttgat atcaaaaact gattttccct ctattatttt | 11880 |
| cgagatttat tttcttaatt ctctttaaca aactagaaat attgtatata caaaaaatta | 11940 |
| taaataatag atgaatagtt taattatagg tgttcatcaa tcgaaaaagc aacgtatctt | 12000 |
| atttaaagtg cgttgctttt ttctcattta taaggttaaa taattctcat atatcaagca | 12060 |
| aagtgacagg cg | 12072 |

SEQ ID NO: 6

| | |
|---|---|
| cccctgatat ttttgactaa accaaatgct aacccagaaa tacaatcact gtgtctaatg | 60 |
| aataatttgt tttataaaca cttttttgtt tacttctcat ttttaattag ttataattaa | 120 |
| ctaaataata gagcattaaa tatatttaat aaaacttatt taatgcaaaa ttatgactaa | 180 |
| catatctata ataaataaag attagatatc aatatattat cgggcaaatg tatcgagcaa | 240 |
| gatgcatcgg atcgatccag gaggtatacc tgattaactt tataaggagg aaaaacatat | 300 |
| gagtatcgtt caagcgaaat ttgaagcaaa agaaacgagt ttccatgtgg aaggatatga | 360 |
| gaagatcgag tacgatttgg tgtatgttga cggcattttt gagatacaaa attctgcgtt | 420 |
| ggcggatgta tatcagggat ttggtagatg cttggcgata gttgatgcga atgtgagtcg | 480 |
| tttgtatggt aatcagatcc aggcgtattt ccaatactat ggtattgagt tacgtttgtt | 540 |
| tcctataacg attacggagc ctgataagac gattcagact ttcgagcgag ttattgacgt | 600 |
| ctttgctgac ttcaagttag tcagaaaaga accagtcttg gtagtgggtg gtggattaat | 660 |
| cacagacgta gtaggcttcg cgtgcagtac ttacagaaga tcttctaact acatccgaat | 720 |
| accgacaacg ttaataggtt tgattgatgc tagtgttgct atcaaggtgg cagtaaatca | 780 |
| cagaaaatta aagaaccgat tgggagctta ccacgcatca cgaaaagtct tcttagactt | 840 |
| ttctttgtta cgaacattgc cgactgatca agtcagaaac ggaatggctg aattagtaaa | 900 |
| gatcgcggtc gtggcacatc aggaggtgtt cgagttgtta gagaagtacg gagaagagtt | 960 |
| attacgaacg catttcggta atatagacgc tactcctgaa attaaggaga tcgcgcaccg | 1020 |
| attgacatac aaagctattc ataagatgtt agagttggag gttcctaact tacacgagtt | 1080 |
| ggacttagac cgtgtaatag cttatggtca tacgtggagt ccgacattag aattggctcc | 1140 |
| tcgtttgcct atgttccacg gacacgcggt caacgtcgat atggcattca gtgcgacgat | 1200 |
| tgctgcacga cgaggttaca ttacgattgc tgagcgtgat agaatcttag gattaatgag | 1260 |
| tcgagtcggt ttaagtttgg accaccctat gttggatatt gacatcttat ggcgaggtac | 1320 |
| agaatctatc actttaactc gagacggatt attgcgtgct gcaatgccga agcctatagg | 1380 |
| cgactgtgta ttcgtcaatg atttgactcg agaagaattg gcagcagcgt tagcggacca | 1440 |
| caaagaatta tgtacgagtt atccgcgtgg tggtgaaggc gtcgatgtct atccagtata | 1500 |
| tcaaaaagag ttaatcggaa gtgtaaagta accc | 1534 |

SEQ ID NO: 7

| | |
|---|---|
| ccctgattaa ctttataagg aggaaaaaca tatgttatca ggacatattg aaggacaaac | 60 |
| tttaaagatg tttgttcata tgacgaaggc taagaaggta ttagaaattg gaatgttcac | 120 |
| aggctactct gcgttagcga tggcagaggc gttaccagag gatggtttgt tagtcgcgtg | 180 |
| cgaagttgat ccttacgcag cggagatagg tcaaaaggca ttccagcaat caccacacgg | 240 |
| cggcaagata cgagttgagt tggacgcagc tttagcgact ttggacaaat tagcagaagc | 300 |
| aggagagagt tttgatttgg ttttcataga cgctgacaag aaggaatacg tggcttattt | 360 |
| tcataagttg ttgggaagtt cattattagc tccagacggt ttcatatgcg ttgacaacac | 420 |
| tttgttgcaa ggagaagtat atttaccggc ggaggagcga tcagtgaatg gtgaggctat | 480 |
| cgcacagttc aatcatactg tcgcaattga tccgcgagtc gagcaagtgt tattgccgtt | 540 |
| gagagatggt ttaactatta ttcgtcgtat tcagccataa ccc | 583 |

SEQ ID NO: 8

| | |
|---|---|
| ccctgattaa ctttataagg aggaaaaaca tatggctcaa tctttaccat tatcttctgc | 60 |
| accagcgact ccgtcattgc catcacagac taagattgct gcgatcatcc agaatatctg | 120 |
| tacgttggcg ttgttgttgt tggctttgcc aattaacgct acgatagtgt ttatcagttt | 180 |
| gttagtattt cgaccacaga aggtgaaggc tgctaaccca caaacaatat taatctcagg | 240 |
| tggaaagatg actaaagcat tgcagttagc acgatctttc catgcagctg gtcatagagt | 300 |
| tgttttggtg gaaacgcata agtattggtt gactggtcac agattctcac aggcagtaga | 360 |
| caaattttat actgtccctg caccgcaaga caatccgcaa gcgtatattc aagctttggt | 420 |
| agacattgtc aaacaggaaa atatagacgt ctatattccg gtgacatctc cggtcggctc | 480 |
| atattacgat tcattggcga agccggaatt gtctcactat tgtgaagtgt ttcacttcga | 540 |
| tgcagacata acacaaatgt tagacgacaa atttgcttta actcaaaaag cacgatcttt | 600 |
| gggcttgtca gtcccgaagt ctttcaaaat aacttctccg gagcaagtaa ttaacttcga | 660 |
| cttctcagga gaaactagaa aatacatctt aaaaagtatt ccatacgaca gtgtacgtcg | 720 |
| tttagatttg acaaagttac cttgtgctac gccggaagag acagcagctt tcgttagaag | 780 |
| tttgccgatc acgccagaga agccgtggat aatgcaggaa tttataccag gcaaggaatt | 840 |
| ttgtacacac tcaactgtca gaaacggaga attacgtttg cactgctgtt gtgagtcatc | 900 |
| agctttccaa gtcaactatg agaacgtaaa caatccgcag ataacagaat gggtccaaca | 960 |
| ttttgtcaaa gagttaaagt tgactggaca aatctctttc gatttcatac aagctgaaga | 1020 |
| tggaacagta tacgctattg agtgtaaccc tcgaactcat tcagcgataa ctactttcta | 1080 |
| tgaccatccg caagttgcag aagcttactt gtcacaggct ccaacaacgg agacaattca | 1140 |
| accattaaca acatctaaac caacttattg gacatatcat gaagtgtgga gattaacggg | 1200 |
| tatccgaagt ttcactcagt tgcaacgatg gttgggcaac atttggcgtg gcactgatgc | 1260 |
| gatttaccaa ccggacgacc cgttaccttt cttgatggtt catcattggc aaattccgtt | 1320 |
| gttattgttg aataatttgc gacgattaaa gggttggaca cgtattgatt tcaacattgg | 1380 |
| aaaattggtc gaattaggag gtgactagcc c | 1411 |

SEQ ID NO: 9

| | |
|---|---|
| ccctgattaa ctttataagg aggaaaaaca tatgcagacg attgatttta atatccgaaa | 60 |
| gttgttagtg gaatggaacg cgacacatcg tgattacgac ttgtcacagt ctttgcacga | 120 |
| attgatagtt gcacaagttg aaagaactcc tgaagctatt gctgttacgt tcgacaagca | 180 |
| gcaattaacg tatcaggaat taaatcataa agcgaaccag ttgggacact acttacaaac | 240 |
| gttaggtgtc caaccggaga cgttagtcgg tgtctgtttg gaacgtagtt tggagatggt | 300 |
| catttgttta ttaggaatat tgaaggcggg cggtgcttat gtccctatcg acccggaata | 360 |
| tcctcaggaa cgtatagctt acatgttaga agactctcag gtgaaggttt tgttgactca | 420 |
| agaaaaatta ttaaatcaga tcccgcacca tcaggcacaa acaatatgtg ttgatagaga | 480 |
| atgggagaaa atctctacac aagcgaatac aaatccgaaa tcaaatatta agacggataa | 540 |
| cttggcatac gtcatttaca cttctggtag tacaggaaaa ccaaaaggtg cgatgaacac | 600 |
| gcataaaggc atatgtaatc gattattgtg gatgcaagag gcttatcaga tcgatagtac | 660 |
| ggacagtatc ttgcaaaaaa cgccgttctc ttttgatgtt tcagtctggg agtttttctg | 720 |
| gacattattg acgggagcgc gattggttat cgctaagcca ggaggccaca aagacagtgc | 780 |
| atatttgata gatttgataa cacaggagca aattacaact ttacactttg tgccgtcaat | 840 |
| gttacaagtc ttcttacaga accgtcacgt aagtaagtgt agttctttaa agcgagtcat | 900 |
| ttgctcagga gaggcgttat caattgattt acagaataga tttttccaac acttgcagtg | 960 |
| tgagttgcac aacttatatg gtccaacaga agctgctatt gacgtaactt tttggcaatg | 1020 |
| tagaaaggat tctaatttaa aatctgtccc gataggtaga cctatagcga atacacaaat | 1080 |
| atatatcttg gacgctgatt tacagccggt taacataggc gtaacgggag agatttatat | 1140 |
| tggaggtgta ggcgtggctc gtggatattt gaataaagag gagttaacta aagaaaaatt | 1200 |
| catcatcaat cctttcccaa actctgaatt caaacgtttg tataaaacgg gcgatttagc | 1260 |
| gcgttattta ccagacggca acatagaata tttgggcaga acagattatc aagtgaaaat | 1320 |
| acgaggctat agaatagaaa taggcgaaat agagaacgtc ttgtctagtc atccacaggt | 1380 |
| gagagaagct gtcgtaatag ctagagatga taacgcgcaa gagaaacaga tcatcgctta | 1440 |
| catcacatat aatagtatca agccgcaatt ggacaactta cgagacttct tgaaggcacg | 1500 |
| tttgccggat tttatgattc ctgcagcttt tgtgatgttg gaacacttac cgttgactcc | 1560 |
| aagtggcaaa gtagatcgaa aggcattgcc taagccagat ttattcaatt actcagaaca | 1620 |
| taattcttat gttgcaccgc gtaatgaagt agaggaaaaa ttggtgcaga tatggtctaa | 1680 |
| cattttacat ttacctaaag taggcgttac tgaaaacttt ttcgcgatcg gtggtaatag | 1740 |
| tttgaaggcg ttacacttaa ttagtcagat tgaggaatta ttcgcaaagg agattagttt | 1800 |
| ggcgacgttg ttgacaaatc cagttattgc ggacttagct aaagttattc aagcgaataa | 1860 |
| ccaaatccac aattcaccat tagtgccgat ccaaccgcag ggcaagcagc aacctttttt | 1920 |
| ctgtatccat ccggcgggtg gacatgtatt atgttacttc aagttggcgc aatacatagg | 1980 |
| aacggaccaa ccattttacg gcttgcaggc tcaaggtttt tacggagatg aagcgccatt | 2040 |
| gacacgtgtt gaggatatgg catctttgta cgtgaaaacg atacgagagt tccaacctca | 2100 |
| aggcccgtac cgagtcggtg gctggtcttt cggcggcgta gtggcgtacg aggtcgcgca | 2160 |
| acaattgcat cgacaaggac aagaggtctc attgttagca attttagata gttatgtgcc | 2220 |
| aatcttattg gataaacaaa agccaatcga tgatgtgtac ttggtgggag tcttgtctcg | 2280 |
| tgtcttcggt ggaatgttcg gtcaagacaa tttagtaaca cctgaagaaa tcgaaaattt | 2340 |
| gacggtagaa gagaaaatca attacatcat tgataaagct agatcagcac gtatttttcc | 2400 |
| tccgggagta gaacgacaga acaatcgtcg tattttggat gtcttagtgg gcactttgaa | 2460 |
| agcaacttat agttacattc gacagcctta tccgggcaag gtcacagttt tccgtgcgcg | 2520 |
| tgaaaaacat ataatggcgc cggacccaac tttggtctgg gttgaattat tttcagttat | 2580 |
| ggcggcgcag gaaataaaga tcatagacgt accgggcaat cactactctt ttgtcttaga | 2640 |
| accacatgta caagtattgg ctcagagatt acaggactgc ttagaaaata actcatgacc | 2700 |
| c | 2701 |

1-7. (canceled)

8. A genetically modified strain of bacteria Escherichia coli Nissle 1917 that produces a material with UV absorbing properties.

9. The genetically modified strain of bacteria of claim 8, wherein the material with UV absorbing properties is a mycosporine-like amino acid.

10. The genetically modified strain of bacteria of claim 8, wherein the mycosporine-like amino acid is shinorine.

11. The genetically modified strain of bacteria of claim 8, comprising the nucleotide sequences shown in SEQ ID NO: 1, SEQ ID NO: 2, SEQ ID NO: 3, and SEQ ID NO: 4.

12. A topical composition comprising the genetically modified strain of bacteria of claim 8, wherein the bacteria is present in the topical composition at a concentration of at least 0.1% by weight of the total composition.

13. A composition comprising the genetically modified strain of bacteria of claim 8 and a sunscreen.

14. A genetically modified strain of commensal bacteria that produces a material with UV absorbing properties, wherein the bacteria comprises a lysis circuit.

15. The genetically modified strain of commensal bacteria of claim 14, wherein the bacteria undergoes lysis in the presence of increased bacterial density and the absence of UV light.
